(19) United States
(12) Patent Application Publication
(10) Pub. No.: US 2026/0146948 A1
(43) Pub. Date: May 28, 2026

(54) ORIGAMI-MEDIATED CHEMICAL AGENT (ORCA) DETECTION PLATFORM

(71) Applicant: BATTELLE MEMORIAL INSTITUTE, Columbus, OH (US)

(72) Inventors: Matthew Neal, Columbus, OH (US); Nickolas R. Andrioff, Columbus, OH (US); Miguel D. Pedrozo, Columbus, OH (US)

(21) Appl. No.: 19/348,726

(22) Filed: Oct. 2, 2025

Related U.S. Application Data
(60) Provisional application No. 63/703,191, filed on Oct. 3, 2024.
(60) Provisional application No. 63/725,376, filed on Nov. 26, 2024.

(51) Int. Cl. G01N 21/64; G01N 33/53

(52) U.S. Cl. CPC G01N 21/6428; G01N 33/5308; G01N 2021/6432

(57) ABSTRACT

Compositions and methods are provided for a deoxyribonucleic acid (DNA) based detection and/or sequestration system and methods of using that system to detect or sequester target molecules. More particularly, a detection composition is disclosed comprising an origami nucleic acid scaffold and a target binding nucleic acid aptamer integrated in the structural scaffold, such that upon binding of the target binding ligand to its target, the origami scaffold produces a signal.

### CROSS REFERENCE TO RELATED APPLICATIONS

This non-provisional application claims the benefit and priority, under 35 U.S.C. § 119(e) and any other applicable laws and statutes, to U.S. Provisional Application Ser. No. 63/703,191 filed on Oct. 3, 2024 and U.S. Provisional Application Ser. No. 63/725,376 filed on Nov. 26, 2024, the entire disclosures of which are incorporated herein by reference.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as a 122,000 byte xml file named “430395.xml,” created on Sep. 25, 2025.

### BACKGROUND

Organophosphorus compounds are extensively used worldwide as pesticides which cause great hazards to human health. Nerve agents, a subcategory of the organophosphorus compounds, have been produced and used during wars, and they have also been used in terrorist activities. More particularly, chemical warfare agents, such as the nerve agents GB and VX, pose a toxic and persistent hazard to conventional military forces and possibly to civilian populations. Organophosphorus compounds interact and inhibit acetylcholinesterase enzyme which leads to the cholinergic crisis and exhibit extraordinary toxicity. A lethal dose can be as little as 0.01 mg of the nerve agent per 1.0 kg of a man's body weight, or 0.70 mg for an average 70 kg man. As such, improved detection tools and sensitive methods of detecting nerve agents will be desirable to both government officials and the general public.

Conventional detection systems suffer a number of disadvantages. For example, enzyme/antibody-based methods suffer from reagent storage and stability issues. Instrument-based methods, such as mass spectrometry, suffer from lack of portability, and high cost of equipment. To overcome these limitations, an improved sensor is desired.

Improvements to the current therapeutics for post-nerve agent exposure are also desired. Current therapies include the administration of drugs that reduce symptoms associated with excessive acetylcholine such as increased secretions (e.g., atropine), inactivation of the acetylcholinesterase enzyme (e.g., 2-PAM), and seizures (e.g., midazolam). The drawbacks to these drugs is that the central nervous system is still impacted by the initial exposure and multiple doses of these drugs are necessary to prevent serious injury. The organophosphate nerve agents remain in the system for hours after the exposure and could also be transferred to others.

The present disclosure is directed to the use of a nucleic acid nanostructure that binds to organophosphates and produces a detectable signal upon binding to the organophosphate.

### SUMMARY

The present disclosure relates to compositions comprising a deoxyribonucleic acid (DNA) based detection and/or sequestration system and methods of using that system to detect or sequester target molecules. More particularly, this disclosure relates to an origami nucleic acid scaffold and a target binding nucleic acid aptamer lock integrated in the structural scaffold, such that upon binding of the target binding ligand to its target, the origami scaffold produces a signal. In one embodiment the origami scaffold comprises donor and acceptor fluorophores that in the absence of the target are in sufficient proximity to one another that the energy emitted from the donor is quenched by the acceptor. Furthermore, upon binding of the target to the target binding ligand, a conformation change is induced in the scaffold, resulting in the efficiency of energy transfer from the donor to the accepter being substantially reduced, or eliminated. In one embodiment the ligand is an aptamer, and in a further embodiment the aptamer specifically binds to an organophosphorus compound, including for example a chemical warfare agent (CWA).

In accordance with one embodiment a nucleic acid nanostructure composition is provided, wherein the composition comprises an origami DNA scaffold, structural staple oligonucleotides and DNA aptamers (oligonucleotides ranging in size from 17-30 base pairs) that can bind organophosphate nerve agents. In one embodiment the structural staple oligonucleotides fold the origami DNA scaffold into a three dimensional space, wherein the shape of the folded origami DNA scaffold is altered upon specific binding of the aptamer to its target organophosphate nerve agent. Detection of this alteration in shape can be diagnostic for the presence of the nerve agent. In one embodiment the nucleic acid nanostructure compositions disclosed herein can be used to sequester the organophosphate to prevent downstream contamination of rescue workers and prevent further injury to exposed individuals.

In one embodiment the DNA nanostructure (aka DNA origami/DNAO) comprises a folded origami DNA scaffold that comprises two domains (optionally in the shape of two planar sheets) linked to each other via a hinge region (similar to a clam shell design or flytrap design). In one embodiment, when the aptamer is bound to an organophosphate nerve agent, a conformational change in the origami scaffold will be induced, resulting in the encapsulation of the nerve agent. The aptamers will ensure binding of the organophosphate nerve agent and prevent interaction of the nerve agent with acetylcholinesterase. Then the sequestered nerve agent will be excreted from the body without inhibiting the acetylcholinesterase enzyme. In an alternative embodiment the DNA nanostructure is further provided with a donor and acceptor fluorescent resonance energy transfer (FRET) pair that upon binding of the organophosphate nerve agent to the aptamer, a conformational change in the origami scaffold will be induced, resulting in the production of a detectable signal.

Nucleic acid nanoparticles (e.g., DNA origami nanostructures) have the advantage of being biocompatible, non-toxic, and can be programmed in many ways. For example, the DNA nanostructure delivery compositions can be programmed to have functional groups that enable them to evade early degradation, that enable them to evade immune responses, and that enable intracellular imaging and targeted and controlled delivery of therapeutic genes. There is no current successful pre-treatment of organophosphate nerve agent exposure. In accordance with one embodiment DNA nanostructures are coated or tagged to increase elimination half-life and therefore, are suitable to be used as a prophylactic treatment for soldiers prior to a high threat mission, or a rescue worker coming to respond to a chemical threat.

### BRIEF DESCRIPTIONS OF THE DRAWINGS

FIG. 1A is a schematic drawing demonstrating the folding of a scaffold DNA using a plurality of staple oligonucleotides to produce two and three dimensional structures. FIG. 1B is a transmission electron microscopy (TEM) image of a scaffold DNA origami cuboids ˜60 nm×25 nm×15 nm formed in accordance with one embodiment of the present disclosure. Various combinations of scaffold DNAs and staple oligonucleotides can be designed to interact in a manner that produces a preselected 2D and 3D DNA origami nanostructures. DNA origami nanostructures can be static or possess range of motion based on conceived design. DNA origami nanostructures can also be designed for the delivery of small molecule therapeutics. DNA origami structures also have the beneficial properties of defined (and uniform) size, geometry, and charge, which can be exploited for tissue tropism. In addition, they can be utilized for spatially controllable drug/reporter loading and functionalization using cell-targeting moieties, and furthermore have excellent biocompatibility and low immunogenicity.

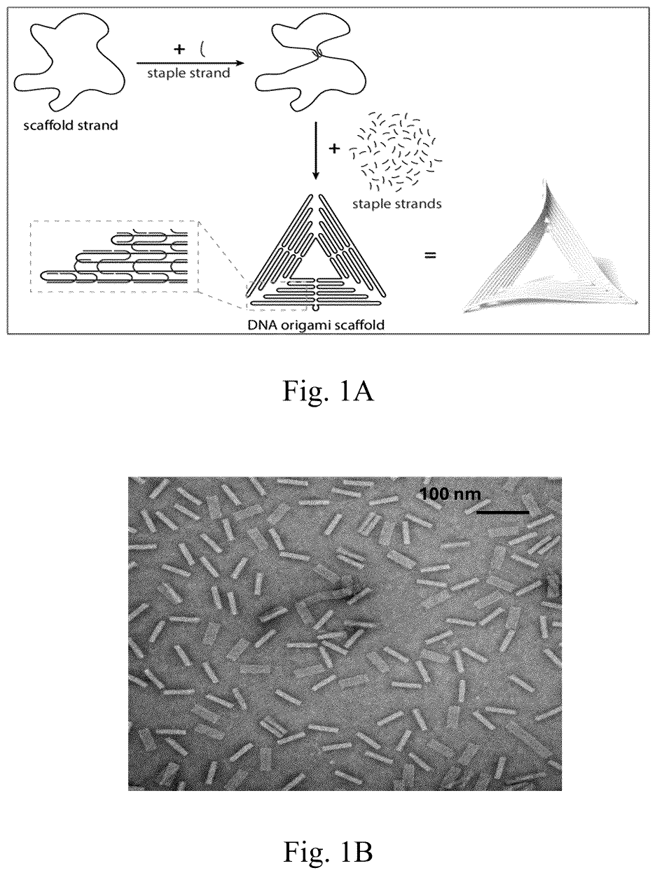

FIG. 2A is a schematic representation of scaffold DNA folded into a clam-shell structure comprising a first and second sheet of origami folded DNA linked to each other via a hinge region, with rows of donor/reporter fluorophores attached to the interior surface of the one sheet of origami folded DNA and rows of acceptor/quencher fluorophores attached to the interior surface of the opposing sheet of origami folded DNA. DNA aptamers that specifically bind to a preselected target(s) are linked to a peripheral edge of a first sheet of origami folded DNA, wherein the aptamers are further provided with a nucleic acid sequence that binds to a nucleic acid sequence present in the second sheet of the origami folded DNA to keep the claim-shell structure in a “closed” orientation until the aptamer binds to its specific target. Binding of the target to the aptamer “opens” the clam-shell to enhance a detectable signal from the donor/reporter fluorophores. FIG. 2B is a schematic representation of signal generation upon specific binding of the target agent to its corresponding aptamer.

FIGS. 3A-3C provide data showing the generation of Origami-mediated Chemical Agents (ORCA). FIG. 3A is a photograph of the results of gel electrophoresis of DNA origami constructs demonstrating the different electrophoretic mobility of various DNA origami constructs. The structures formed are reproducibly generated nanostructures which are dependent on salt conditions. As shown in FIGS. 3B and 3C these structures demonstrate both closed (FIG. 3B) and open (FIG. 3C) functionality.

FIGS. 4A & 4B provide data showing the addition of fluorophores and aptamers to the ORCA scaffold. FIG. 4A shows optimal fold conditions identified with demonstration of fluorophore and aptamer addition. FIG. 4B demonstrates the DNAO aptamer lock sequences with broad organophosphate binding can be used to substantially reduce signal relative to scaffold alone.

FIGS. 5A & 5B provides data showing the increase in fluorescence upon contact of the ORCA signaling construct with an organophosphate (OP), in this instance profenofos. FIG. 5A presents the data as a bar graph showing the detection of Cy3 fluorescence in the absence of the target compound (profenofos), with the target compound and a control without the presence of a quencher fluorophore. Contact of the ORCA signaling construct with an organophosphate results in the opening of the closed claim-shell configured folded DNA structure, resulting in an increase in detectable fluorescence. FIG. 5B presents the data in standard graph form.

FIG. 6 is a schematic drawing demonstrating the use of guide staple oligonucleotides to stabilize the clam-shell configuration in a locked position to assist the interaction of the locking sequence of the aptamers to bind to their complementary sequences. Once the locking sequence of the aptamers have engaged with their complementary sequences, the guide staple oligonucleotides can be removed via contact with guide staple remover oligonucleotides, wherein the guide staple remover oligonucleotides are the exact complement of the guide staple nucleic acids that are bound to the DNA scaffold. After removal of the guide staples the composition is ready for use in detecting organophosphates. In one embodiment the guide staples are positioned at ˜⅓ the length of the opposing DNA origami sheets from the hinge region.

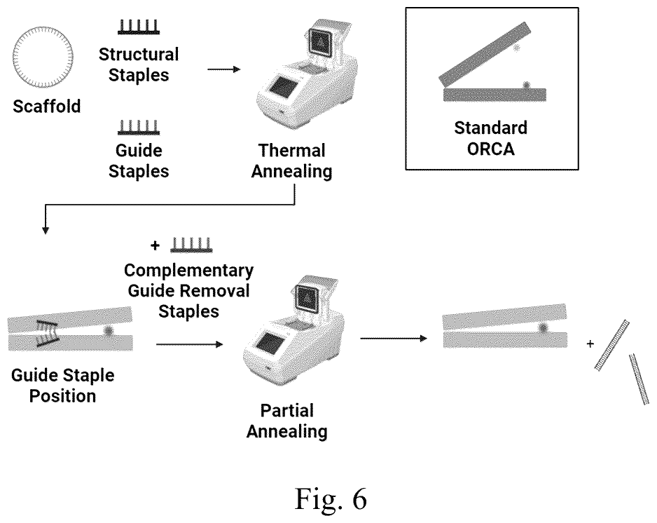

FIG. 7 presents data demonstrating that the use of guide staple oligonucleotides to prepare the signaling ORCAs having improved signal to noise ratios and improved signal detection upon binding to an organophosphate.

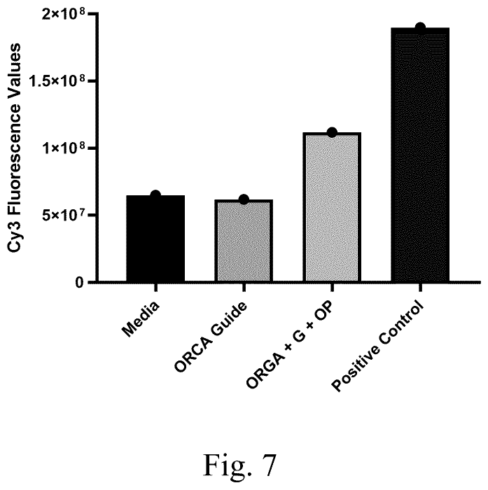

FIG. 8 provides exemplified guide staple oligonucleotide for use in accordance with the present disclosure. The guide staples have been split into a structural component and a guide staple component (indicated by the inclusion of PolyT and polyAs). Fully complementary staples (C1-c and D12-c) should be introduced to successfully remove the guide staples C1-a and D12-a.

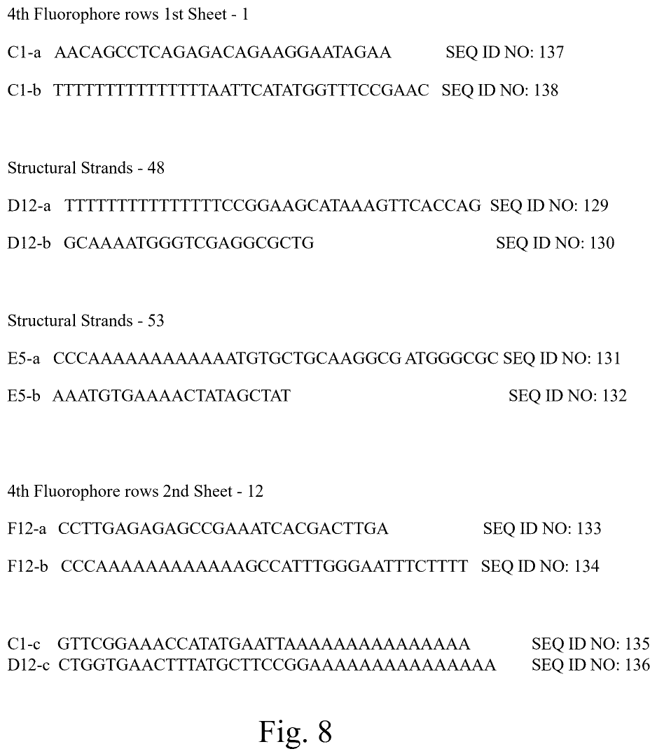

### DETAILED DESCRIPTION

### Definitions

As used herein, the term “complementary base pairing” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.” As used herein the term “complementarity” when used in the context of a nucleic acid sequence, defines a level of sequence identity between two nucleic acid sequences that allows for specific hybridization between the two respective sequences.

As used herein the term “DNA scaffold” defines a large single stranded sequence of DNA of approximately 500 to about 31,000 bases that can be folded by a plurality of preselected complementary DNA staple oligonucleotides into a predetermined two dimensional or three dimensional shape.

As used herein the term “staple oligonucleotide” or “structural staple oligonucleotide” defines a single stranded nucleic acid sequence that together with other staple oligonucleotides will self-assemble with a single stranded DNA scaffold to reversibly fold the single stranded DNA scaffold into a predetermined compacted 2-D and 3-D structure. Staple oligonucleotides typically comprise two or more nucleic acid sequences that are complementary, optionally having at least 80%, 90% or 95% sequence identity, to non-contiguous sequences present in a DNA scaffold, wherein the staple oligonucleotide sequences sharing complementarity with the DNA scaffold are linked to one another via a linking nucleic sequence, optionally wherein the linking nucleic acid sequence lacks complementarity with the DNA scaffold.

As used herein the term “guide staple oligonucleotide” or “guide staple” defines a single stranded nucleic acid sequence that will self-assemble with an origami folded DNA scaffold and will stabilize the preexisting two and/or three dimensional shape of the folded DNA scaffold.

As used herein the term “guide staple remover oligonucleotide” or “guide staple remover” defines a single stranded nucleic acid sequence that shares 100% complementarity with its corresponding guide staple oligonucleotide wherein the guide staple remover functions to disrupt binding of the guide staple to the DNA scaffold.

As used herein the term “aptamer” defines a relatively short nucleic acids sequence of 10 to 50 nucleotides that will bind a specific target molecule, or family of target molecules.

As used herein the term “organophosphorus compound” defines a class of compounds comprising phosphorus linked to carbon atoms, typically esters of phosphoric acid that can be mono esters, di esters or tri esters depending on the number of attached organic groups. Included in this class are organophosphate nerve agents such as Sarin (GB), Soman (GD), Tabun (GA), and VX.

As used herein a “signal” is any detectable change in reflected or emitted light within the electromagnetic spectrum. In one embodiment a signal can be light emitted by a fluorescent molecule.

### EMBODIMENTS

In accordance with one embodiment, a DNA origami nanostructure construct is designed, wherein the nanostructure construct comprises a DNA scaffold and a plurality of staple oligonucleotides that fold the DNA scaffold into a non-native, predetermined two or three dimensional shape. The nanostructure construct is further provided with a DNA aptamer that specifically binds to a target compound, including for example an organophosphorus compound, wherein the aptamer is integrated into a peripheral edge of the folded DNA scaffold of the DNA origami nanostructure construct. In one embodiment the aptamer is linked to the DNA scaffold via partial or full complementary ssDNA base pair binding to one or more sequences of the DNA scaffold. In one embodiment, detection of the target moiety is realized by identification of a change in shape of the folded DNA scaffold upon binding of the target moiety to the aptamer. In one embodiment a detectable signal is generated upon binding of the preselected target moiety to the aptamer. In one embodiment the signal generated is a fluorescent signal or alteration in a fluorescent signal. In one embodiment the signal generated is a colorimetric signal or alteration in a colorimetric signal. In one embodiment the signal is generated as a result of the target moiety binding to the aptamer of the DNA origami nanostructure construct and inducing an alteration of the shape of the folded DNA.

In one embodiment the designed DNA origami nanostructure construct is further provided with a marker that produces a detectable signal upon binding of the aptamer with its target moiety (e.g., an organophosphate compound). In one embodiment the detectable signal is a change in fluorescence emitted from a fluorophore linked to the DNA scaffold of the DNA origami nanostructure. In one embodiment the DNA origami nanostructure is provided with a donor fluorophore and an acceptor fluorophore of a Fluorescence Resonance Energy Transfer (FRET) pair wherein in the absence of the target moiety (e.g., an organophosphorus compound) the donor and acceptor fluorophores are within sufficient proximity to allow for resonance energy transfer from the donor fluorophore to the acceptor fluorophore, and upon binding of the target moiety (e.g., an organophosphorus compound) to the aptamer, the shape of the DNA scaffold is altered in a manner that disrupts (i.e., decreases or eliminates) resonance energy transfer from the donor fluorophore to the acceptor fluorophore, resulting in an increase in detectable donor fluorescence.

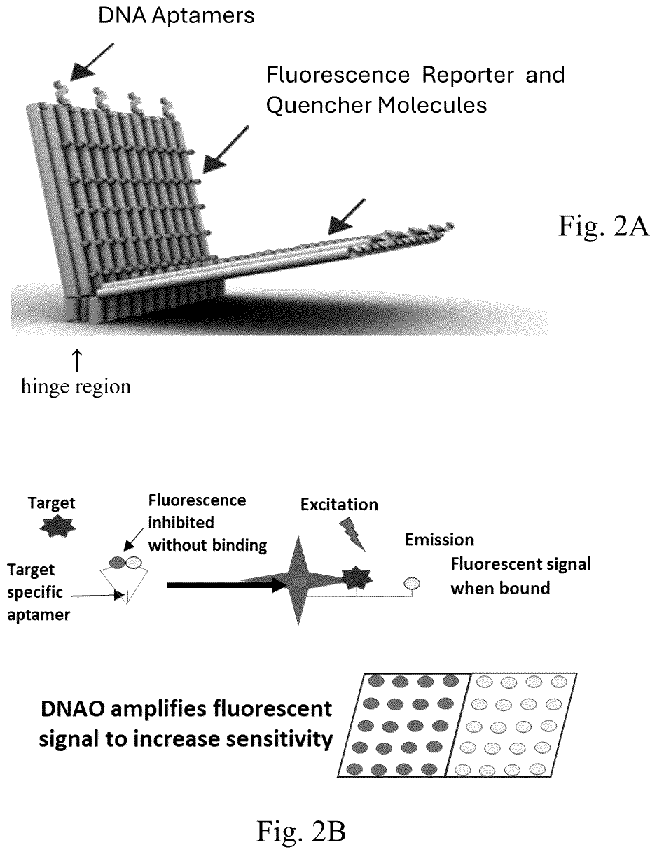

In accordance with one embodiment the DNA scaffold of the DNA origami nanostructure is folded by the staple oligonucleotides to form a first nucleic acid arm and a second nucleic acid arm, wherein the first and second arms are linked to each other in a “hinge” region (see FIG. 2A), allowing the two arms to flex in a clamshell motion. In one embodiment, one or more acceptor fluorophores are linked to the first arm of the DNA origami nanostructure, one or more corresponding donor fluorophores are linked to the second arm, and a plurality of target specific binding aptamers are linked to the peripheral edge of the first and second nucleic acid arm of the folded DNA scaffold. In this embodiment the aptamer is releasably linked to the first and second arm of the folded DNA scaffold to hold the first and second arms of the folded DNA scaffold in an orientation that places the acceptor fluorophores and the donor fluorophores in sufficient proximity to allow fluorescent energy transfer from the donor fluorophore to the acceptor fluorophore. Upon binding of the target moiety to the aptamer, the aptamer is released from at least one arm of the DNA scaffold, and the DNA scaffold changes shape resulting in a decrease or elimination of fluorescent energy transfer from the donor fluorophore to the acceptor fluorophore. Thus, detecting an alteration in fluorescence from the donor and/or acceptor fluorophore is an indicator of the presence of the target moiety in a sample that is contacted with the DNA origami nanostructure construct. In one embodiment the target moiety is an organophosphorus compound, and the aptamer is specifically designed to bind to the the organophosphorus compound and be released from the DNA scaffold upon binding to the organophosphorus compound.

In accordance with one embodiment, a DNA scaffold and a set of staple oligonucleotides are provided wherein the staple oligonucleotides are designed to fold the DNA scaffold into two distinct planar structures that are linked together at a first end of each of the first and second planar structures, wherein the first and second planar structures are free to move relative to one other in a clamshell motion, such that the distance between the second ends of the planar structures, opposite of the joined first ends of the first and second planar structures, can be increased or decreased. In one embodiment, the opposing interior surface of the first and second planar structures are populated with signal generating entities that produce a detectable signal when the first and second planar structures are moved from a closed configuration to an open configuration. In one embodiment one or more acceptor fluorophores are linked to the opposing interior surface of the first planar structures of the DNA origami nanostructure, and one or more corresponding donor fluorophores are linked to the opposing interior surface of the second planar structure. In one embodiment, the acceptor and donor fluorophores are positioned on the respective opposing surfaces of the first and second planar structures at the distal end of the first and second planar structures relative to the joined first ends of the first and second planar structures. In one embodiment the acceptor and donor fluorophores are positioned on the respective opposing surfaces of the first and second planar structures at the distal ⅓rd end portion of the first and second planar structures relative to the joined first ends of the first and second planar structures. In one embodiment the acceptor and donor fluorophores are positioned on the respective opposing surfaces of the first and second planar structures at the distal ¼th end portion of the first and second planar structures relative to the joined first ends of the first and second planar structures. In one embodiment the acceptor and donor fluorophores are positioned on the respective opposing surfaces of the first and second planar structures at the distal 1/10th end portion of the first and second planar structures relative to the joined first ends of the first and second planar structures.

In one embodiment, a DNA scaffold and a set of staple oligonucleotides are provided wherein the staple oligonucleotides are designed to fold the DNA scaffold into two distinct planar structures that are linked together at a first end of each of the first and second planar structures, wherein the first and second planar structures are reversibly biased in an “open” configuration where a signal is produced. In embodiments having acceptor fluorophores linked to the opposing interior surface of the first planar structure of the DNA origami nanostructure, and donor fluorophores linked to the opposing interior surface of the second planar structure, the open configuration is defined as a distance between the first and second planar interior surfaces sufficient to disrupt or prevent fluorescence resonance energy transfer from the donor fluorophore to the acceptor fluorophore.

In one embodiment the DNA scaffold is folded into two distinct planar structures that are linked together at a first end of each of the first and second planar structures, wherein one or more aptamers hold the two planar structures of the DNA scaffold in a conformation that alters the signal that is generated when the the first and second planar structures are in the open configuration. In one embodiment one or more aptamers are releasably linked to the first and second planar structures along the peripheral edge of the first and second planar structures, wherein linkage of the aptamers to the DNA scaffold holds the DNA scaffold in the closed position. In one embodiment, the one or more aptamers are linear constructs where a first end of the aptamer is linked to the end portion of the first planar structure that is distal to the first end, and the second end of the aptamer is linked to the end portion of the second planar structure that is distal to the first end, wherein at least one of the linked ends of the aptamer is releasable upon contact with a target moiety. In one embodiment, the aptamer is covalently linked to the peripheral edge of the first planar structure and is releasably linked to the peripheral edge of the second planar structure. In one embodiment, the aptamer is releasably linked to the peripheral edge of the first and second planar structure.

In accordance with one embodiment the aptamer comprises a first nucleic acid sequence that is complementary to a sequence of the DNA scaffold, and/or one of the staple oligomers of the construct, present on the peripheral edge of the first planar structure of the DNA origami nanostructure, and a second nucleic acid sequence that is complementary to a sequence of the DNA scaffold, and/or one of the staple oligomers of the construct, present on the peripheral edge of the second planar structure of the DNA origami nanostructure, wherein said aptamer further comprises a target specific region that interacts with a target moiety, wherein interaction between the aptamer target specific region and the target causes the release of at least one end of the aptamer from the DNA origami nanostructure/staple oligomers of the construct, and results in an increase in distance between the first and second planar structures and the generation of a detectable signal.

In accordance with one embodiment the aptamer is an organophosphorus compound specific aptamer, comprising a nucleic acid region that specifically binds to organophosphorus compounds, wherein the aptamer further comprises a first nucleic acid sequence that is complementary to a sequence of the DNA scaffold, and/or one of the staple oligomers of the construct, present on the peripheral edge of the first planar structure of the DNA origami nanostructure and a second nucleic acid sequence that is complementary to a sequence of the DNA scaffold, and/or one of the staple oligomers of the construct, present on the peripheral edge of the second planar structure. In one further embodiment one or more acceptor fluorophores are linked to the opposing interior surface of the first planar structures of the DNA origami nanostructure, and one or more corresponding donor fluorophores are linked to the opposing interior surface of the second planar structure. In accordance with this embodiment, in the absence of the organophosphorus compound the donor and acceptor fluorophores are within sufficient proximity to allow for resonance energy transfer from the donor fluorophore to the acceptor fluorophore and the fluorescent signal from the donor fluorophore is quenched. Upon binding of the organophosphorus compound to the organophosphorus compound specific aptamer linked to the DNA origami nanostructure, the binding of the aptamer to either, or both the first and second arms of the DNA origami nanostructure is disrupted, the first and second planar structures move apart from each other and a fluorescent signal from the the donor fluorophore is detectable or significantly increased and the fluorescent signal from the acceptor fluorophore is decreased.

In accordance with one embodiment the aptamers used in the DNA origami nanostructures disclosed herein are selected from those disclosed in Table 1:

TABLE 1

| SEQ | | Sequence with underlined sequence representing sequences |
|---|---|---|
| ID | Sequence | that share partial complementarity to a DNA staple |
| NO | Name | sequence present on the peripheral edge of the construct |
| 1 | Sensor | GTATGTTAAAAAACAGGGAAGCAGCCTTTACAGAGAGAATAAAAAAAGCTCTGTGG |
| | Lock v1- | |
| | 1 | |
| 2 | Sensor | GTAATTTAATATGTAAATGCTGGGCTTAGGTTGGGTTATATAAAAAAGCTCTGTGG |
| | Lock v1- | |
| | 2 | |
| 3 | Sensor | ATTTTGCGGAGCCAGCAGCAAACGCCTGCAACAGTGCCACGAAAAAAGCTCTGTGG |
| | Lock v1- | |
| | 3 | |
| 4 | Sensor | TACTATGGCCAACGTCAAAGGGTCCACTATTAAAGAACGTGAAAAAAGCTCTGTGG |
| | Lock v1- | |
| | 4 | |
| 5 | Sensor | AGCTTGCTGCAGCGATTCTTGATCGCCACAGAGCTTTTTTTTCGCATTAAATTTTTGTT |
| | Lock v1- | CGCTTCT |
| | 5 | |
| 6 | Sensor | AGCTTGCTGCAGCGATTCTTGATCGCCACAGAGCTTTTTTGAAGCAAAGCGGATTGC |
| | Lock v1- | ATGCGAACGAGTAGATATTAGA |
| | 6 | |
| 7 | Sensor | AGCTTGCTGCAGCGATTCTTGATCGCCACAGAGCTTTTTTTTTTCATGAGGAAGTTTC |
| | Lock v1- | CGTGTACAGACCAGGGCTGAC |
| | 7 | |
| 8 | Sensor | AGCTTGCTGCAGCGATTCTTGATCGCCACAGAGCTTTTTTAGAATGGAAAGCGCAGT |
| | Lock v1- | CTCCCGGAATAGGTGTCAGGAG |
| | 8 | |
| 9 | Sensor | GTATGTTAAAAAACAGGGAAGCAGCCTTTACAGAGAGAATAGGAGCTCTGTGG |
| | Lock v2- | |
| | 1 | |
| 10 | Sensor | GTAATTTAATATGTAAATGCTGGGCTTAGGTTGGGTTATATGGAGCTCTGTGG |
| | Lock v2- | |
| | 2 | |
| 11 | Sensor | ATTTTGCGGAGCCAGCAGCAAACGCCTGCAACAGTGCCACGGGAGCTCTGTGG |
| | Lock v2- | |
| | 3 | |
| 12 | Sensor | TACTATGGCCAACGTCAAAGGGTCCACTATTAAAGAACGTGGGAGCTCTGTGG |
| | Lock v2- | |
| | 4 | |
| 13 | Sensor | AGCTTGCTGCAGCGATTCTTGATCGCCACAGAGCTCCTTCGCATTAAATTTTTGTTCG |
| | Lock v2- | CTTCT |
| | 5 | |
| 14 | Sensor | AGCTTGCTGCAGCGATTCTTGATCGCCACAGAGCTCCGAAGCAAAGCGGATTGCATG |
| | Lock v2- | CGAACGAGTAGATATTAGA |
| | 6 | |
| 15 | Sensor | AGCTTGCTGCAGCGATTCTTGATCGCCACAGAGCTCCTTTTCATGAGGAAGTT |
| | Lock v2- | TCCGTGTACAGACCAGGGCTGAC |
| | 7 | |
| 16 | Sensor | AGCTTGCTGCAGCGATTCTTGATCGCCACAGAGCTCCAGAATGGAAAGCGCAGTCTC |
| | Lock v2- | CCGGAATAGGTGTCAGGAG |
| | 8 | |

In accordance with one embodiment the DNA scaffold of the DNA origami nanostructure is an M13 genomic sequence and the staples designed to fold the DNA scaffold into a three dimensional clam-shell configuration are selected from Table 2:

TABLE 2

| SEQ ID | Sequence | |
|---|---|---|
| NO | Name | Sequence |
| 17 | Structural | CAACGCTGTCCAGATAAACCAGTTTTAGAATCCAA |
| | Strands-1 | |
| 18 | Structural | ACAACGCAATAAGATATTTTCTCAGATAAAATAGC |
| | Strands-2 | |
| 19 | Structural | TTAGTATACGCGCCCAATAATAAATCAATAATTTG |
| | Strands-3 | |
| 20 | Structural | TCATCTTAAATTCTAACATGTTATCATTTGCGGGA |
| | Strands-4 | |
| 21 | Structural | AAAGAACAATTGAGGACAAAACGAGAACGGTATTC |
| | Strands-5 | |
| 22 | Structural | TCATCAAAACAATACATTTAAAGTGAATTCAAATA |
| | Strands-6 | |
| 23 | Structural | GCGGAATAAAATCGAAGAAGAAACCTCCATGCAAA |
| | Strands-7 | |
| 24 | Structural | ATGGAAGGTCAGATGTACATAAACATAGATGGTTT |
| | Strands-8 | |
| 25 | Structural | TTACAAATTTGGATAGTACCTTTACCTTAAGACGC |
| | Strands-9 | |
| 26 | Structural | CGTTATTTGATTATTTGCTTTTCAAGAAGGTCTGA |
| | Strands- | |
| | 10 | |
| 27 | Structural | ACTATCGCACCAGTGAACCACACCCTCAGTATTAA |
| | Strands- | |
| | 11 | |
| 28 | Structural | AGCCATTGACGCTCTTAACACTGAAAAAGAGTAAC |
| | Strands- | |
| | 12 | |
| 29 | Structural | TTGATTACAGAGATCGAACTGAAGGAATAGGATTT |
| | Strands- | |
| | 13 | |
| 30 | Structural | AGGAACGTGCCTGATAAAAGGTCGCCATAGTTGGC |
| | Strands-14 | |
| 31 | Structural | GAGCGGGATCCAGAGATTATTACAGAGGTTGCTGA |
| | Strands-15 | |
| 32 | Structural | AAATCAGATTGTAAAAGGGTG |
| | Strands- | |
| | 16 | |
| 33 | Structural | TGTACCCATAAATTTGTAATACAATTCTTGTITTA |
| | Strands- | |
| | 17 | |
| 34 | Structural | ATTGTATCAGTCAATAAAAATTAGCTATTTCCATA |
| | Strands- | |
| | 18 | |
| 35 | Structural | GCCTTTACGCGAGCGTACGGTTTTTAATTTAAACA |
| | Strands- | |
| | 19 | |
| 36 | Structural | TATATTTGCAAATGCAATTCTCAAAAAGAGGTCTT |
| | Strands- | |
| | 20 | |
| 37 | Structural | GTACCAACATTAACATATAATCCAACAGGCGGAAT |
| | Strands- | |
| | 21 | |
| 38 | Structural | GAACCAGATTGAATCGTTTACTCAGGACAGTGAAT |
| | Strands- | |
| | 22 | |
| 39 | Structural | CGAAAGAAATGACCAGGAATTAACGGAAGGATATT |
| | Strands- | |
| | 23 | |
| 40 | Structural | GAGCAACTCTACGTACGTAACAATCATACCTAAAA |
| | Strands- | |
| | 24 | |
| 41 | Structural | ATGCAGAGTAGAAAAGAGTAAATGAACGATTAAAC |
| | Strands- | |
| | 25 | |
| 42 | Structural | AACCAAAAGAACTGCGAGAAAACCTGCTTTTGACC |
| | Strands-26 | |
| 43 | Structural | CGGAACGGAATACACCGCTTITTATCAG |
| | Strands-27 | |
| 44 | Structural | ACCAACTATGCCACTCGGAACTCCAAAA |
| | Strands- | |
| | 28 | |
| 45 | Structural | CCTCAGCGGAGCCTATTTTCTGAGCCACCTCAGTA |
| | Strands- | |
| | 29 | |
| 46 | Structural | CTACAGAAATTTTTAGCGGAGCCGCCACAGTATAG |
| | Strands- | |
| | 30 | |
| 47 | Structural | AGGCTTGGAATTTCACGATCTACCCATGCTGAGAC |
| | Strands- | |
| | 31 | |
| 48 | Structural | TTTCCAGGGGATAGATTAGGATAAGTTT |
| | Strands- | |
| | 32 | |
| 49 | Structural | TAAACAACCCTCAGGCCGTCGGCGTCAT |
| | Strands- | |
| | 33 | |
| 50 | Structural | AAATTATTCATTAAAACCGCCTACCGAAGAAACAA |
| | Strands- | |
| | 34 | |
| 51 | Structural | GTTTAGTATGAGAATAGAAAGGGAATTG |
| | Strands- | |
| | 35 | |
| 52 | Structural | CTGAATTACAAACACCTTTAGACCATCGATAGCAGCACCGTACAGAAT |
| | Strands- | |
| | 36 | |
| 53 | Structural | AATCCTGTGCCCTTCACCGCCTCGCTCA |
| | Strands- | |
| | 37 | |
| 54 | Structural | GAAAATACATACATTACGCA |
| | Strands-38 | |
| 55 | Structural | CAATAGCAAGCAAAATCGTA |
| | Strands-39 | |
| 56 | Structural | AATTACCTGAGCAACGCAGA |
| | Strands- | |
| | 40 | |
| 57 | Structural | AATACCTACATTTTGCAACA |
| | Strands- | |
| | 41 | |
| 58 | Structural | TGCCAGCTGCATTACTTTCCAGTCGGGA |
| | Strands- | |
| | 42 | |
| 59 | Structural | AGATTCAAACGTTAATATTTTG |
| | Strands- | |
| | 43 | |
| 60 | Structural | TTCAACTATACCCTGACTATTA |
| | Strands- | |
| | 44 | |
| 61 | Structural | CGAATAATGGCTTTGAGGACTA |
| | Strands- | |
| | 45 | |
| 62 | Structural | CAAGTTTGAATAAATCCTCATT |
| | Strands- | |
| | 46 | |
| 63 | Structural | CAATTCCACACAACAGCTGATTTTGATGGCTAAATCAGCGAAATGAGAAG |
| | Strands- | |
| | 47 | |
| 64 | Structural | CCGGAAGCATAAAGTTCACCAGGCAAAATGGGTCGAGGCGCTG |
| | Strands- | |
| | 48 | |
| 65 | Structural | CCTGGGGTGCCTAATGCGCCAGAAGAATATCACCCACACGCTGCCGATTA |
| | Strands- | |
| | 49 | |
| 66 | Structural | GCTAACTCACATTAAGAGAGGCTGAGTGTGATGGCCACCCGCC |
| | Strands-50 | |
| 67 | Structural | TGCGCTCACTGCCCGATGAATCACAAGAGCGAAAAAGCTACAGGTGCTTT |
| | Strands-51 | |
| 68 | Structural | TAACAACCCACGTTGGTGTAGATTAAGTTGGGTAAC |
| | Strands-52 | |
| 69 | Structural | TGTGCTGCAAGGCGATGGGCGCAAATGTGAAAACTATAGCTAT |
| | Strands- | |
| | 53 | |
| 70 | Structural | CGCCAGCTGGCGAAATCTGCCATAGCCAG |
| | Strands- | |
| | 54 | |
| 71 | Structural | GTGCGGGCCTCTTCGCGACAGTAATTCGCAAAAGCCGATATTC |
| | Strands- | |
| | 55 | |
| 72 | Structural | AACTGTTGGGAAGGGGATCGCAAATAGGA |
| | Strands- | |
| | 56 | |
| 73 | Structural | GGTGCCGAAGCGCCATTCGCCATTCAGGCCGGCAC |
| | Strands- | |
| | 57 | |
| 74 | Biotin | TCAACCGATTGAGGGAG |
| | Anchors-1 | |
| 75 | Biotin | GCTATCTTCCCTCAGAGC |
| | Anchors-2 | |
| 76 | Biotin | AACCTTGCTTCTGTGAAATAAAGATGGTTTAA |
| | Anchors-3 | |
| 77 | Biotin | TTCCTGTAGTCACGACGT |
| | Anchors-4 | |
| 78 | Biotin | AACAAGATTCTCCGGGTTTGCAGAGAGTTGCAGCAAGC |
| | Anchors-5 | |
| 79 | Biotin | CGCCTGAGGGCTTGAGAAATTAATTATTTGCACGTAAA |
| | Anchors-6 | |
| 80 | Foot-1 | AATTTTAATCCCATACAAACTGTAGCATTCCACAGGTTGCGC |
| 81 | Foot-2 | AAACACCCGACAATCATCGCCCACGCATGAAACAATAATTAA |
| 82 | Foot-3 | GGAATCAATAAGGCGTTAAATTCGCTATAGTACAAGTATCAT |
| 83 | Foot-4 | TAATAGATTGCCAGTAGTAAAATGTTTATAATTGCTTTTGAACGTAAGA |
| 84 | Foot-5 | GAGCACTACAATATTCCTTTTTCATTTTTGCGGAT |
| 85 | Foot-6 | ATACGTGCCATCACGAATTAGGCAATAAAGCCTCATATCAGG |
| 86 | Foot-7 | CGAACGTGATTTAGAGCTTGACCACGCTTGGGAAC |
| 87 | Foot-8 | ATTGACCGTAATGGTTTTCCCGTGAAAT |
| 88 | Foot-9 | AGGCAAAGCAAATTCACCGAGTAAAAGAAAGCCGGTCATTGCTGGAGCA |
| 89 | Foot-10 | ACTTTAATCATTGTAGAAGTTTTAGAGCTCTTTAG |
| 90 | Foot-11 | TGAAATAATCTTACCCCCTGCAACCTATTATTCTG |
| 91 | Foot-12 | CACCAGTCCTAATTTCCTGAACAAGAAA |
| 92 | Foot-13 | CCCTCAGAACCGCCTTAATGCCAACGCTCAGCTAC |
| 93 | Removable | AACGAGCAGAGCAAGCCCTTTGCGACAT |
| | Hinge-1 | |
| 94 | Removable | GTGATAATAATTACAGATAAGTACGAGCTTGCACC |
| | Hinge-2 | |
| 95 | Removable | CGTCAATATATCAAGCGTAGAAGTGAATTTTTCCC |
| | Hinge-3 | |
| 96 | Removable | GTGAGGCAACCGTTCTGAAAGTGGCTATTAAAATA |
| | Hinge-4 | |
| 97 | Removable | TGTTATCGGCCCTGCCCAGCAAGCCCCCGGCGAGA |
| | Hinge-5 | |
| 98 | Removable | GCCAGGGGATAGGTCGTCGGA |
| | Hinge-6 | |
| 99 | Removable | GAATCGACAAAGGCGAGCATAACAGGCAGGCTTAG |
| | Hinge-7 | |
| 100 | Removable | GTACCTTGACTGGATTGCAAAGAATTACGTAAATT |
| | Hinge-8 | |
| 101 | Removable | TAAATTGCAAGCGCAACCGATACCGATA |
| | Hinge-9 | |
| 102 | Removable | ACAGCCCGTTTCGTAAACATGTAAACAG |
| | Hinge-10 | |
| 103 | Removable | ACCCTCACCACCGGAGGTGAA |
| | Hinge-11 | |

In one embodiment fluorophores are attached to oligonucleotides that share complementarity with sequences of the scaffold as a means of linking fluorophores to specific locations on the folded DNA scaffold. In one embodiment the donor fluorophore is Cyanine3 (Cy3) or Cyanine5 (Cy5) and the corresponding acceptor/quencher fluorophore is a Black Hole Quencher (BHQ) dye, including for example, 3′ Black Hole Quencher®-2 or 3′ Black Hole Quencher®-3. In one embodiment the donor and acceptor fluorophores are covalently linked to an oligonucleotide as designated in Table 3:

TABLE 3

| SEQ | | | |
|---|---|---|---|
| ID NO | Name | Sequence | Fluorophore |
| 104 | 1st Fluorophore rows 1st | GCATTAGTCCTTATAAAGGTGGCAACATATAA | Donor |
| | Sheet-1 | AAGGGCATG | |
| 105 | 1st Fluorophore rows 1st | ATTAAGACACGGGAGAATTAACAACGTC | Donor |
| | Sheet Blank-1 | | |
| 106 | 1st Fluorophore rows 1st | AAAAATGATAGAAGGCTTATCCAAGCAA | Donor |
| | Sheet Blank-2 | | |
| 107 | 1st Fluorophore rows 1st | GCCGTTTTGAATATAAAGTACCAATCGC | Donor |
| | Sheet Blank-3 | | |
| 108 | 1st Fluorophore rows 1st | CATATTTATCCAATCGCAAGACGAGACT | Donor |
| | Sheet Blank-4 | | |
| 109 | 1st Fluorophore rows 1st | ACCTTTTTTGATGAAACAAACAGAATAC | Donor |
| | Sheet Blank-5 | | |
| 110 | 1st Fluorophore rows 1st | CAAGTTACTATCATCATATTCCAATTTT | Donor |
| | Sheet Blank-6 | | |
| 111 | 1st Fluorophore rows 1st | AAAAGTTTTCTAAAGCATCACCTGAGGC | Donor |
| | Sheet Blank-7 | | |
| 112 | 1st Fluorophore rows 1st | GGTCAGTAAATCGTCTGAAATGACAATA | Donor |
| | Sheet Blank-8 | | |
| 113 | 1st Fluorophore rows 1st | TTACCGCCCCTCGTTAGAATCAGCGCTT | Donor |
| | Sheet Blank-9 | | |
| 114 | 1st Fluorophore rows 1st | GCATTAGTCCTTATAAAGGTGGCAACATATAA | Donor |
| | Sheet Blank-10 | AAGGGCATG | |
| 115 | 1st Fluorophore rows 1st | AATGCGCCCCGTCTATCAGGGCTGTTCC | Donor |
| | Sheet Blank-11 | | |
| 116 | 1st Fluorophore rows 1st | AGTTTGGAGGCCAACGCGCGGGTTGCGT | Donor |
| | Sheet Blank-12 | | |
| 117 | 1st Fluorophore rows 2nd | TTTTAACCCTCCAGCCAGCTTTCTGCGC | Acceptor |
| | Sheet Blank-1 | | |
| 118 | 1st Fluorophore rows 2nd | GCCGGAGAAAGCAAATATTTAACTCATT | Acceptor |
| | Sheet Blank-2 | | |
| 119 | 1st Fluorophore rows 2nd | AACCTGTTTTTTAGAACCCTCAAGAAAG | Acceptor |
| | Sheet Blank-3 | | |
| 120 | 1st Fluorophore rows 2nd | AGGAAGCCTAACAGTTGATTCCGTCAAT | Acceptor |
| | Sheet Blank-4 | | |
| 121 | 1st Fluorophore rows 2nd | CATAGTAAGTTCAGAAAACGAGCTTCAA | Acceptor |
| | Sheet Blank-5 | | |
| 122 | 1st Fluorophore rows 2nd | CAAGAACCCAACATTATTACAGTACATA | Acceptor |
| | Sheet Blank-6 | | |
| 123 | 1st Fluorophore rows 2nd | AATACGTATTGAAAGAGGACAGTCTTGA | Acceptor |
| | Sheet Blank-7 | | |
| 124 | 1st Fluorophore rows 2nd | TGAAAATCGAGGGTAGCAACGGGGGTAA | Acceptor |
| | Sheet Blank-8 | | |
| 125 | 1st Fluorophore rows 2nd | AACCGCCACTTTCAACAGTTTCTCACGT | Acceptor |
| | Sheet Blank-9 | | |
| 126 | 1st Fluorophore rows 2nd | TCCAGTAAAGAGGGTTGATATACCTCAG | Acceptor |
| | Sheet Blank-10 | | |
| 127 | 1st Fluorophore rows 2nd | ACTGTAGCTGGCCTTGATATTCTACCGT | Acceptor |
| | Sheet Blank-11 | | |
| 128 | 1st Fluorophore rows 2nd | AGACGATGCGTTTTGCCGGAAACGTCACCAATG | Acceptor |
| | Sheet Blank-12 | AACGTCAG | |

In one embodiment background fluorescence from the DNA origami nanostructure complex is reduced by use of guide staples. Guide staples are oligonucleotides that share complementarity with the DNA scaffold and assist in stabilizing the DNA origami nanostructure complex in a “closed” formation, during storage and prior to exposure of the nanostructure complex to samples that may contain an organophosphate. Guide staples are used to stabilize the conformation of the DNA scaffold to allow the aptamers sufficient time to integrate and fully engage their complementary sequences and hold the clam-shell configuration in a closed orientation. Once the aptamers are fully integrated into the folded DNA scaffold, the guide staples can be removed to enhance the sensitivity of the DNA origami nanostructure complex to detect a target moiety (e.g., an organophosphorus compound). In one embodiment the guide staples are removed through the use of guide removal staple oligonucleotides, wherein the guide removal staple oligonucleotides have 100% sequence identity to the guide staple oligonucleotides. Accordingly, in one embodiment, once the clam-shell configuration of the origami structure is stabilized in a closed configuration and the aptamers are fully integrated, the composition is contacted with guide staple remover oligonucleotides in a partial annealing process to remove the guide staple oligonucleotides prior to use of the DNA origami nanostructure complex to detect the target moiety. The use of guide staple oligonucleotides and guide removal staple oligonucleotides are used to reduce background signal and amplify the signal (i.e., enhance the signal to noise ratio) and enhance the sensitivity and speed of detection.

In one embodiment the DNA origami nanostructure complex is part of a device that can be used as a wearable detection device. In one embodiment the device comprises multiple DNA origami nanostructure complexes combined on a single platform, optionally attached to a surface for wearable detection device, wherein the multiple DNA origami nanostructure complexes differ from one another based on the target binding ligand (e.g. aptamer) and the florescent signal produced upon binding of the target moiety to the target binding ligand.

In accordance with one embodiment a wearable, sensitive and broad chemical warfare agent (CWA) detection platform is provided, comprising an active fluorescent reporter that provides a reproducible and reliable signal only when in contact with the target organophosphate or threat agent. In one embodiment the DNA nanostructure detection compositions described herein can be applied to surfaces to provide a passive early detection system.

In various embodiments, the DNA nanostructure detection compositions described herein may comprise any any DNA nanostructure delivery composition, such as DNA origami structures. In one embodiment the DNA scaffold comprises an M13 bacteriophage based single stranded DNA.

In one embodiment, a DNA nanostructure detection composition is provided. A DNA nanostructure composition (e.g., a DNA origami structure), as a detection platform, is programmable and offers an opportunity for precise scale-up and manufacturing. In this embodiment, the biologic and non-viral nature of the DNA nanostructure delivery composition reduces the chance of adverse immune reactions for embodiments related to treatment or remediation of organophosphate nerve agent exposure. In this embodiment, control of each nucleotide that forms a part of the DNA nanostructure delivery composition (e.g., DNA origami nanostructure) allows for the precise design and modification of the structure. In this embodiment, the DNA nanostructure delivery composition can undergo self-base pairing (i.e., a DNA origami structure) to fold into structures that can form the single-stranded DNA scaffold that can encapsulate a target moiety that binds to a target binding ligand (optionally a DNA aptamer) that is covalently linked to the origami DNA scaffold.

In another embodiment, the DNA nanostructure delivery composition can comprise i) a single stranded DNA scaffold and ii) one or more oligonucleotides that bind through complementary base pairing with a segment of the DNA scaffold, wherein the one or more oligonucleotides cause the DNA scaffold to fold. In this embodiment, the one or more oligonucleotides can comprise overhangs that bind through complementary base paring with the nucleic acid aptamers designed to bind a target moiety. In this embodiment, the overhangs can be located within a cavity within the DNA scaffold, and the cavity can be covered by a lid and a hinge allowing the bound target moiety to be completely enclosed within the cavity when the lid is shut. In this embodiment, the lid can further comprise oligonucleotide strands that bind through complementary base pairing with other oligonucleotide strands attached to the DNA scaffold when the lid is in the closed position. DNA nanostructure delivery compositions (e.g., DNA origami structures) are described in U.S. Pat. No. 9,765,341, incorporated herein by reference.

As used herein, the term “complementary base pairing” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

In one embodiment the DNA nanostructure compositions disclosed herein can be linked to a solid surface, optionally wherein the solid surface comprises two or more different DNA nanostructure compositions that differ by the aptamer associated with the DNA scaffold. In one embodiment biotinylated ssDNA overhangs can be included in the hinge portion of the DNA scaffold for attachment to a solid surface (i.e., SEQ ID NOs: 74-79). In any of the DNA nanostructure compositions described herein, the DNA scaffold and the one or more oligonucleotides can comprise M13 bacteriophage DNA.

In one embodiment, the compositions described herein may be formulated as pharmaceutical compositions for parenteral or topical administration. Such pharmaceutical compositions and processes for making the same are known in the art for both humans and non-human mammals. See, e.g., Remington: The Science and practice of pharmacy, (1995) A. Gennaro, et al., eds., 19th ed., Mack Publishing Co. Additional active ingredients may be included in the compositions.

In one aspect, the nucleic acid nanostructure composition may be administered, for example, directly into the blood stream of a patient, into muscle, into an internal organ, or can be administered in a topical formulation. In various embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but they may be more suitably formulated as a sterile non-aqueous solution or as a dried form to be used in conjunction with a suitable vehicle such as sterile, pyrogen-free water or sterile saline. The preparation under sterile conditions, by lyophilization to produce a sterile lyophilized powder for a parenteral formulation, may readily be accomplished using standard pharmaceutical techniques well-known to those skilled in the art. In one embodiment, the solubility of the composition used in the preparation of a parenteral formulation may be increased by the use of appropriate formulation techniques, such as the incorporation of solubility-enhancing agents.

In one illustrative embodiment, pharmaceutical compositions for parenteral administration comprise: a) a pharmaceutically active amount of the nucleic acid nanostructure; b) a pharmaceutically acceptable pH buffering agent to provide a pH in the range of about pH 4.5 to about pH 9; c) an ionic strength modifying agent in the concentration range of about 0 to about 300 millimolar; and d) water soluble viscosity modifying agent in the concentration range of about 0.25% to about 10% total formula weight or any combinations of a), b), c) and d) are provided.

In various illustrative embodiments, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and nonionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. Typically, non-acidic viscosity enhancing agents, such as a neutral or a basic agent are employed in order to facilitate achieving the desired pH of the formulation.

In one embodiment, the solubility of the compositions described herein used in the preparation of a parenteral formulation may be increased by the use of appropriate formulation techniques, such as the incorporation of solubility-enhancing agents.

In other embodiments, the compositions described herein may be administered topically. A variety of dose forms and bases can be applied to the topical preparations, such as an ointment, cream, gel, gel ointment, plaster (e.g. cataplasm, poultice), solution, powders, and the like. These preparations may be prepared by any conventional method with conventional pharmaceutically acceptable carriers or diluents as described below.

For example, vaseline, higher alcohols, beeswax, vegetable oils, polyethylene glycol, etc. can be used. In the preparation of a cream formulation, fats and oils, waxes, higher fatty acids, higher alcohols, fatty acid esters, purified water, emulsifying agents etc. can be used. In the preparation of gel formulations, conventional gelling materials such as polyacrylates (e.g. sodium polyacrylate), hydroxypropyl cellulose, hydroxypropyl methyl cellulose, polyvinyl alcohol, polyvinylpyrrolidone, purified water, lower alcohols, polyhydric alcohols, polyethylene glycol, and the like are used. In the preparation of a gel ointment, an emulsifying agent (preferably nonionic surfactants), an oily substance (e.g. liquid paraffin, triglycerides, and the like), etc. are used in addition to the gelling materials as mentioned above. A plaster such as cataplasm or poultice can be prepared by spreading a gel preparation as mentioned above onto a support (e.g. fabrics, non-woven fabrics). In addition to the above-mentioned ingredients, paraffins, squalane, lanolin, cholesterol esters, higher fatty acid esters, and the like may optionally be used. Moreover, antioxidants such as BHA, BHT, propyl gallate, pyrogallol, tocopherol, etc. may also be incorporated. In addition to the above-mentioned preparations and components, there may optionally be used any other conventional formulations for incorporation with any other additives.

Applicant has demonstrated successful and reproducible production of the ORCA nanostructure with a FRET reporter and a specific DNA aptamer as a “lock”; Exposure of ORCA to an organophosphate pesticide as a surrogate for nerve agents for the “key” led to dynamic motion of the structure and positive fluorescence generation. Addition of a guide staple enhanced closing of the base ORCA structure to a minimum, while maintaining function with the OP aptamer “lock”.

The ORCA nanostructure has functionality built-in for ease of attachment onto surfaces. The DNA aptamers for use in the present invention are very specific and considered equivalent or better than monoclonal antibodies. Origami DNA exhibits exceptional stability compared to RNA and proteins and the ORCA nanostructure complexes disclosed herein can be used in conjunction with a range of reporter elements, including colorimetric and electrochemical signaling. Furthermore, the modular nature of the ORCA nanostructure complexes allows for expansion of target threat detection and multi-plex detection of target agents.

### EXEMPLIFIED EMBODIMENTS

In accordance with embodiment 1, a DNA nanostructure composition is provided wherein the composition comprises: i) a single stranded DNA scaffold and ii) an aptamer linked to said DNA scaffold, wherein the aptamer specifically binds to an organophosphorus compound, further wherein the the binding of the organophosphorus compound to the target binding ligand causes a conformation change to the single stranded DNA scaffold resulting in the production of a detectable signal.

In accordance with embodiment 2, the DNA nanostructure composition of embodiment 1 is provided wherein the detectable signal is the production or alteration in a fluorescent signal.

In accordance with embodiment 3, the DNA nanostructure composition of embodiment 1 or 2 is provided wherein the aptamer specifically binds to an organophosphate compound selected from an organophosphate nerve agent or an organophosphate pesticide.

In accordance with embodiment 4, the DNA nanostructure composition of any one of embodiments 1-3 is provided wherein the organophosphorus compound is an organophosphate nerve agent selected from the group consisting of Sarin (GB), Soman (GD), Tabun (GA) and VX.

In accordance with embodiment 5, the DNA nanostructure composition of any one of embodiments 1˜4 is provided wherein said aptamer comprises a sequence selected from the group consisting of SEQ ID Nos 1-16, optionally said aptamer comprises a sequence selected from the group consisting of SEQ ID Nos 1-8.

In accordance with embodiment 6, the DNA nanostructure composition of any one of embodiments 1-5 is provided wherein said single stranded DNA scaffold is folded into a three dimensional shape by a plurality of staple oligonucleotides, wherein the three dimensional shape comprises a first and second sheet of scaffold DNA joined to one another via a hinge region extending along one edge of the respective first and second sheets, said hinge region allowing for relative movement of the first and second sheets to alter the distance between the opposing surface of the first and second sheets;

said aptamer being linked to a free edge of said first sheet, optionally the edge opposite said hinge region, wherein the aptamers comprise a locking sequence that binds to a sequence located on the second sheet of the single stranded DNA scaffold.

In accordance with embodiment 7, the DNA nanostructure composition of any one of embodiments 1-6 is provided wherein the scaffold DNA comprises single stranded M13 bacteriophage genomic DNA folded into a three dimensional shape using the staple oligonucleotides of SEQ ID Nos: 17-73.

In accordance with embodiment 8, the DNA nanostructure composition of any one of embodiments 1-7 is provided wherein a plurality of aptamers that differ in structure are linked to the edge of said first sheet, optionally wherein the plurality of aptamers comprises 3 to 8 aptamers independently selected from SEQ ID Nos: 1-8.

In accordance with embodiment 9, the DNA nanostructure composition of any one of embodiments 1-8 is provided wherein the single stranded DNA scaffold comprises a donor fluorophore and an acceptor fluorophore of a FRET pair wherein in the absence of an organophosphorus compound bound to the aptamer said acceptor fluorophore is in sufficient proximity to said donor fluorophore that the fluorescence from the donor fluorophore is quenched by the acceptor fluorophore and the detectable signal from the donor fluorophore is increased upon binding of the organophosphorus compound to the DNA aptamer, optionally wherein the acceptor and donor fluorophores are attached to oligonucleotides that share complementarity with sequences of the scaffold as a means of linking fluorophores to specific locations on the folded DNA scaffold.

In accordance with embodiment 10, the DNA nanostructure composition of any one of embodiments 1-9 is provided wherein said first sheet of the single stranded DNA scaffold comprises a plurality of donor fluorophores and said second sheet of the single stranded DNA scaffold comprises a plurality of acceptor fluorophores.

In accordance with embodiment 11, the DNA nanostructure composition of any one of embodiments 1-10 is provided wherein the donor fluorophore is covalently linked to the 3′ terminus of an oligonucleotide selected from the group consisting of SEQ ID Nos: 104-116 and the accepter fluorophore is covalently linked to the 3′ terminus of an oligonucleotide selected from the group consisting of SEQ ID Nos: 117-128.

In accordance with embodiment 12, the DNA nanostructure composition of any one of embodiments 1-11 is provided wherein the donor fluorophore is Cyanine3 (Cy3) and the acceptor fluorophore is a Black Hole Quencher fluorophore.

In accordance with embodiment 13, the DNA nanostructure composition of any one of embodiments 1-12 is provided wherein the DNA nanostructure composition is covalently linked to a solid support.

In accordance with embodiment 14, the DNA nanostructure composition of embodiment 13 is provided wherein said solid support comprises a plurality of said DNA nanostructure compositions that differ from one one other based on the specificity of the aptamer for a specific organophosphate and/or the signal produced by the individual DNA nanostructure composition.

In accordance with embodiment 15, a method of detecting an organophosphorus compound is provided, wherein the method comprises
contacting a sample with a DNA nanostructure composition of any one of embodiments 1-13; and
scanning the sample for fluorescence after contacting the sample with said origami nucleic acid scaffold, wherein the detection of an increase in donor fluorophore fluorescence indicates the presence of the target moiety in the sample.

In accordance with embodiment 16, a method of sequestering an organophosphorus compound is provided, said method comprising contacting an organophosphorus compound with an origami nucleic acid scaffold comprising a
an aptamer that specifically binds to an organophosphorus compound, said aptamer being linked to said origami nucleic acid scaffold, wherein binding of the organophosphorus compound to said aptamer causes a conformation change in the origami nucleic acid scaffold that sequesters the organophosphorus compound within, or closely associated with, the origami nucleic acid scaffold.

### Example 1

Generation of ORCA Nanostructure with OP DNA Aptamer and Guide Staples

ORCA nanostructures were synthesized by combining water, 10×TAE, either 120 or 200 mM MgCl₂, 7560 nucleotide length M13 ssDNA scaffold, and ssDNA staple strands in a 1:10 ratio of scaffold to staples. Structures were then annealed with a temperature ramp of 65° C. for 20 minutes, then decreasing temperature by 0.1° C./6 min until the mixture reached 40° C. Following the thermal annealing process, structures were purified using 100 kDa Amicon filters. Filters were washed with 500 μL of 1×TAE buffer with 12 mM or 20 mM MgCl₂, respective to each sample, and centrifuged at 1800 RCF for 9 minutes. Samples were then loaded onto the filters and spun at 1800 RCF for 9 minutes, washed with 500 μL of annealing buffer, and eluted by inverting the Amicon filters into collection tubes and centrifuging at 10000 RCF for 2 minutes. Guide staples are added at a ratio of 1250:1 for subsequent removal of the guide staple, removal staples were added to the the purified samples comprising guide staple: structures, and then the purified sample underwent a partial annealing process by incubating at 35 degrees C. for 2 hrs and then allowed to cool to 4 deg C.

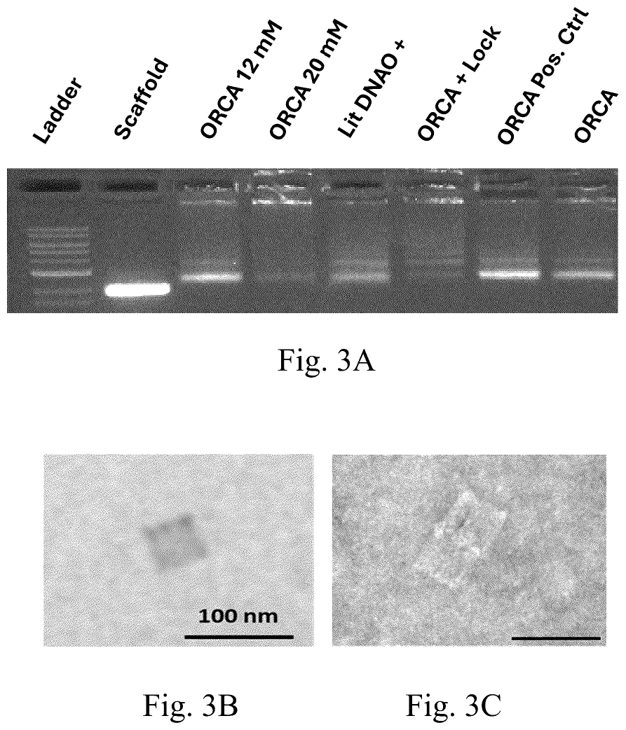

Following production of the ORCA nanostructure, visualization of the structures was performed by transmission electron microscopy (TEM) where the structures were diluted in respective annealing buffer to 10 nM. Formvar coated copper grids were plasma cleaned to remove any debris, and 10 uL of sample was deposited onto a grid for 2 minutes. Sample was wicked off using filter paper and the grid was then incubated with a 2% aqueous uranyl acetate solution for 30 seconds. Excess stain was removed via filter paper and samples were allowed to dry. Imaging was performed on an FEI Tecnai G2 Spirit TEM (See FIGS. 3B & 3C).

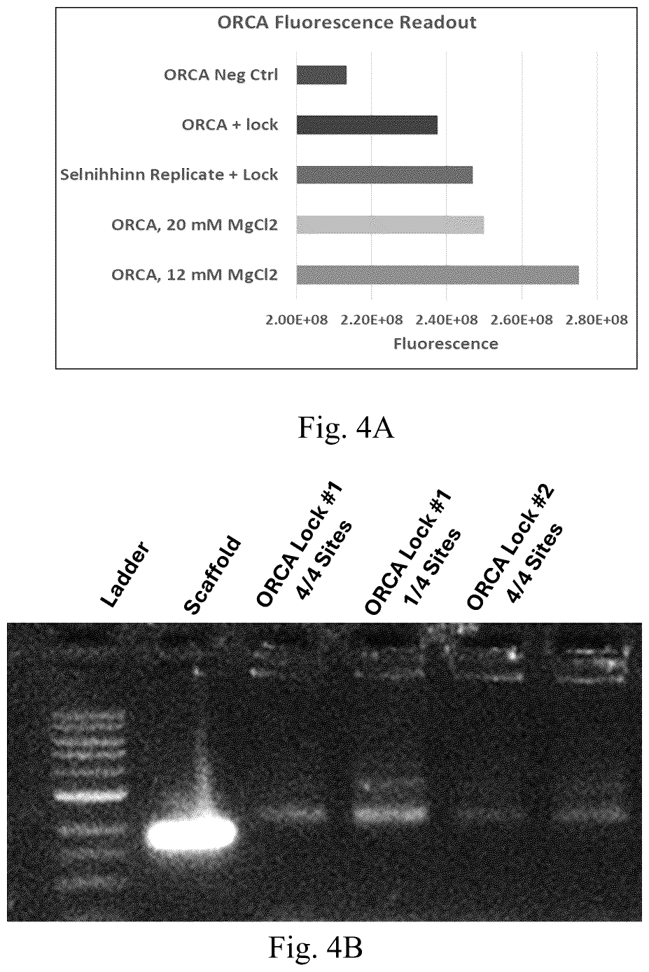

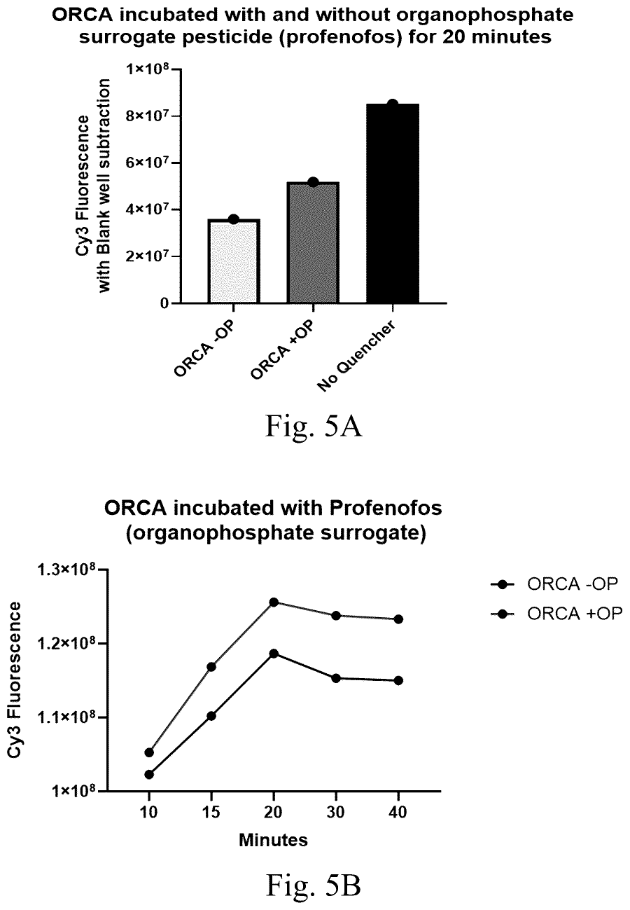

To confirm production of the ORCA structures and evaluate fluorescence following profenofos addition, profenofos was prepared at 10 μM by diluting 1 μL stock into 99 μL PBS, and 4 μL of profenofos was added to respective ORCA samples. The DNA nanostructure samples were then diluted to 20 nM, stained with blue loading dye, and loaded onto a 1% agarose gel. The gel was prepared with 1.2 g of UltraPure agarose in 1×TAE buffer, and stained with 5 μL of Ethidium bromide. The samples migrated at 90 V for 90 minutes while the gel rig was half-submerged in a cool water containing an ice pack. Samples were then imaged using a BioRad GelDoc XR system. Results are shown in FIGS. 4B, 5A and 5B).

For fluorescent quantification via spectrophotometer, ORCA samples were prepared at 80 nM and 50 uL samples were added to respective wells of a Corning black half-area clear bottom 96-well plate. Baseline fluorescence was recorded using a Spectramax i3 spectrophotometer with 550 nm excitation and 570 nm emission wavelength. Profenofos was prepared at 10 μM by diluting 1 μL stock into 99 μL PBS, and 4 μL of profenofos was added to respective samples on the 96 well plate. Fluorescence measurements were taken at approximately 1 minute after dosing and again every 5 minutes for the first 30 minutes following the initial dose.

1. A DNA nanostructure composition comprising: i) a single stranded DNA scaffold and ii) an aptamer linked to said DNA scaffold, wherein the aptamer specifically binds to an organophosphorus compound, wherein the the binding of the organophosphorus compound to the aptamer causes a conformation change to the single stranded DNA scaffold resulting in the production of a detectable signal.

2. The DNA nanostructure composition of claim 1 wherein the detectable signal is the production or alteration in a fluorescent signal.

3. The DNA nanostructure composition of claim 1 wherein the organophosphorus compound is an organophosphate nerve agent or an organophosphate pesticide.

4. The DNA nanostructure composition of claim 1 wherein the organophosphorus compound is an organophosphate nerve agent selected from the group consisting of Sarin (GB), Soman (GD), Tabun (GA) and VX.

5. The DNA nanostructure composition of claim 1 further comprising a plurality of staple oligonucleotides that fold said DNA scaffold into a predetermined shape.

6. The DNA nanostructure composition of claim 1 wherein said aptamer comprises a sequence selected from the group consisting of SEQ ID Nos 1-16, optionally said aptamer comprises a sequence selected from the group consisting of SEQ ID Nos 1-8.

7. The DNA nanostructure composition of claim 1 wherein said single stranded DNA scaffold is folded into a three dimensional shape by a plurality of staple oligonucleotides, wherein the three dimensional shape comprises a first and second sheet of scaffold DNA joined to one another via a hinge region extending along one edge of the respective first and second sheets, said hinge region allowing for relative movement of the first and second sheets to alter the distance between the opposing surface of the first and second sheets;
said aptamer being linked to a free edge of said first sheet, optionally the edge opposite said hinge region, wherein the said aptamers comprise a locking sequence that releasably binds to a sequence located on the second sheet of the single stranded DNA scaffold.

8. The DNA nanostructure composition of claim 7 wherein a plurality of aptamers that differ in structure are linked to the edge of said first sheet, optionally wherein the plurality of aptamers comprises 3 to 8 aptamers independently selected from SEQ ID Nos: 1-8.

9. The DNA nanostructure composition of claim 7 wherein the single stranded DNA scaffold comprises a donor fluorophore and an acceptor fluorophore of a FRET pair wherein in the absence of a target moiety bound to the target binding moiety said acceptor fluorophore is in sufficient proximity to said donor fluorophore that the fluorescence from the donor fluorophore is quenched by the acceptor fluorophore and the detectable signal from the donor fluorophore is increased upon binding of the target moiety to the aptamer.

10. The DNA nanostructure composition of claim 7 wherein said first sheet of the single stranded DNA scaffold comprises a plurality of donor fluorophores and said second sheet of the single stranded DNA scaffold comprises a plurality of acceptor fluorophores.

11. The DNA nanostructure composition of claim 10 wherein the DNA nanostructure composition is covalently linked to a solid support.

12. The DNA nanostructure composition of claim 11 wherein said solid support comprises a plurality of said DNA nanostructure compositions that differ from one one other based on the specificity of the aptamer for a specific organophosphate and/or the signal produced by the individual DNA nanostructure composition.

13. A DNA nanostructure composition comprising:
i) a single stranded DNA scaffold;
ii) a plurality of staple oligonucleotides;
iii) an aptamer that specifically binds to a target moiety;
iv) a donor and acceptor fluorophore of a fluorescence resonance energy transfer (FRET) pair, wherein said staple oligonucleotides are designed to fold the DNA scaffold into two distinct planar structures that are linked together at a first end of each of the first and second planar structures, wherein the first and second planar structures are free to move relative to one other in a clamshell motion, and the opposing interior surface of the first and second planar structures are populated with said donor and acceptor fluorophores, respectively;
said aptamer is releasably linked to the first and second planar structures along a peripheral edge of the first and second planar structures, to hold the DNA scaffold in a conformation that allows for FRET transfer between the donor and acceptor fluorophores, said aptamer designed to be released from binding to both the first and second planar structures upon binding to the target moiety.

14. DNA nanostructure composition of claim 13 wherein said aptamer specifically binds to an organophosphorus compound and is released from the DNA scaffold upon binding to an organophosphorus compound.

15. A method of detecting an organophosphorus compound, said method comprising
contacting a sample with the DNA nanostructure composition of claim 1; and
scanning the sample for fluorescence after contacting the sample with said DNA nanostructure, wherein the detection of an increase in donor fluorophore fluorescence indicates the presence of the organophosphorus compound in the sample.

16. A method of detecting an organophosphorus compound, said method comprising
contacting a sample with the DNA nanostructure composition of claim 13; and
scanning the sample for fluorescence after contacting the sample with said DNA nanostructure, wherein the detection of an increase in donor fluorophore fluorescence indicates the presence of the organophosphorus compound in the sample.

17. A method of sequestering an organophosphorus compound, said method comprising contacting an organophosphorus compound with an origami nucleic acid scaffold comprising
an aptamer that specifically binds to an organophosphorus compound, said aptamer being linked to said origami nucleic acid scaffold, wherein binding of the organophosphorus compound to said aptamer causes a conformation change in the origami nucleic acid scaffold that sequesters the organophosphorus compound within, or closely associated with, the origami nucleic acid scaffold.


SEQUENCE LISTING (138 sequences)

SEQ ID NO: 1 - DNA, 56 bp, synthetic construct
  source 1..56: mol_type = other DNA
gtatgttaaaaaacagggaagcagcctttacagagagaataaaaaaagctctgtgg

SEQ ID NO: 2 - DNA, 56 bp, synthetic construct
  source 1..56: mol_type = other DNA
gtaatttaatatgtaaatgctgggcttaggttgggttatataaaaaagctctgtgg

SEQ ID NO: 3 - DNA, 56 bp, synthetic construct
  source 1..56: mol_type = other DNA
attttgcggagccagcagcaaacgcctgcaacagtgccacgaaaaaagctctgtgg

SEQ ID NO: 4 - DNA, 56 bp, synthetic construct
  source 1..56: mol_type = other DNA
tactatggccaacgtcaaagggtccactattaaagaacgtgaaaaaagctctgtgg

SEQ ID NO: 5 - DNA, 66 bp, synthetic construct
  source 1..66: mol_type = other DNA
agcttgctgcagcgattcttgatcgccacagagcttttttttcgcattaaatttttgttcgcttct

SEQ ID NO: 6 - DNA, 79 bp, synthetic construct
  source 1..79: mol_type = other DNA
agcttgctgcagcgattcttgatcgccacagagcttttttgaagcaaagcggattgcatgcgaacgagtagatattaga

SEQ ID NO: 7 - DNA, 79 bp, synthetic construct
  source 1..79: mol_type = other DNA
agcttgctgcagcgattcttgatcgccacagagcttttttttttcatgaggaagtttccgtgtacagaccagggctgac

SEQ ID NO: 8 - DNA, 79 bp, synthetic construct
  source 1..79: mol_type = other DNA
agcttgctgcagcgattcttgatcgccacagagcttttttagaatggaaagcgcagtctcccggaataggtgtcaggag

SEQ ID NO: 9 - DNA, 53 bp, synthetic construct
  source 1..53: mol_type = other DNA
gtatgttaaaaaacagggaagcagcctttacagagagaataggagctctgtgg

SEQ ID NO: 10 - DNA, 53 bp, synthetic construct
  source 1..53: mol_type = other DNA
gtaatttaatatgtaaatgctgggcttaggttgggttatatggagctctgtgg

SEQ ID NO: 11 - DNA, 53 bp, synthetic construct
  source 1..53: mol_type = other DNA
attttgcggagccagcagcaaacgcctgcaacagtgccacgggagctctgtgg

SEQ ID NO: 12 - DNA, 53 bp, synthetic construct
  source 1..53: mol_type = other DNA
tactatggccaacgtcaaagggtccactattaaagaacgtgggagctctgtgg

SEQ ID NO: 13 - DNA, 63 bp, synthetic construct
  source 1..63: mol_type = other DNA
agcttgctgcagcgattcttgatcgccacagagctccttcgcattaaatttttgttcgcttct

SEQ ID NO: 14 - DNA, 76 bp, synthetic construct
  source 1..76: mol_type = other DNA
agcttgctgcagcgattcttgatcgccacagagctccgaagcaaagcggattgcatgcgaacgagtagatattaga

SEQ ID NO: 15 - DNA, 76 bp, synthetic construct
  source 1..76: mol_type = other DNA
agcttgctgcagcgattcttgatcgccacagagctccttttcatgaggaagtttccgtgtacagaccagggctgac

SEQ ID NO: 16 - DNA, 76 bp, synthetic construct
  source 1..76: mol_type = other DNA
agcttgctgcagcgattcttgatcgccacagagctccagaatggaaagcgcagtctcccggaataggtgtcaggag

SEQ ID NO: 17 - DNA, 35 bp, synthetic construct
  source 1..35: mol_type = other DNA
caacgctgtccagataaaccagttttagaatccaa

SEQ ID NO: 18 - DNA, 35 bp, synthetic construct
  source 1..35: mol_type = other DNA
acaacgcaataagatattttctcagataaaatagc

SEQ ID NO: 19 - DNA, 35 bp, synthetic construct
  source 1..35: mol_type = other DNA
ttagtatacgcgcccaataataaatcaataatttg

SEQ ID NO: 20 - DNA, 35 bp, synthetic construct
  source 1..35: mol_type = other DNA
tcatcttaaattctaacatgttatcatttgcggga

SEQ ID NO: 21 - DNA, 35 bp, synthetic construct
  source 1..35: mol_type = other DNA
aaagaacaattgaggacaaaacgagaacggtattc

SEQ ID NO: 22 - DNA, 35 bp, synthetic construct
  source 1..35: mol_type = other DNA
tcatcaaaacaatacatttaaagtgaattcaaata

SEQ ID NO: 23 - DNA, 35 bp, synthetic construct
  source 1..35: mol_type = other DNA
gcggaataaaatcgaagaagaaacctccatgcaaa

SEQ ID NO: 24 - DNA, 35 bp, synthetic construct
  source 1..35: mol_type = other DNA
atggaaggtcagatgtacataaacatagatggttt

SEQ ID NO: 25 - DNA, 35 bp, synthetic construct
  source 1..35: mol_type = other DNA
ttacaaatttggatagtacctttaccttaagacgc

SEQ ID NO: 26 - DNA, 35 bp, synthetic construct
  source 1..35: mol_type = other DNA
cgttatttgattatttgcttttcaagaaggtctga

SEQ ID NO: 27 - DNA, 35 bp, synthetic construct
  source 1..35: mol_type = other DNA
actatcgcaccagtgaaccacaccctcagtattaa

SEQ ID NO: 28 - DNA, 35 bp, synthetic construct
  source 1..35: mol_type = other DNA
agccattgacgctcttaacactgaaaaagagtaac

SEQ ID NO: 29 - DNA, 35 bp, synthetic construct
  source 1..35: mol_type = other DNA
ttgattacagagatcgaactgaaggaataggattt

SEQ ID NO: 30 - DNA, 35 bp, synthetic construct
  source 1..35: mol_type = other DNA
aggaacgtgcctgataaaaggtcgccatagttggc

SEQ ID NO: 31 - DNA, 35 bp, synthetic construct
  source 1..35: mol_type = other DNA
gagcgggatccagagattattacagaggttgctga

SEQ ID NO: 32 - DNA, 21 bp, synthetic construct
  source 1..21: mol_type = other DNA
aaatcagattgtaaaagggtg

SEQ ID NO: 33 - DNA, 35 bp, synthetic construct
  source 1..35: mol_type = other DNA
tgtacccataaatttgtaatacaattcttgtttta

SEQ ID NO: 34 - DNA, 35 bp, synthetic construct
  source 1..35: mol_type = other DNA
attgtatcagtcaataaaaattagctatttccata

SEQ ID NO: 35 - DNA, 35 bp, synthetic construct
  source 1..35: mol_type = other DNA
gcctttacgcgagcgtacggtttttaatttaaaca

SEQ ID NO: 36 - DNA, 35 bp, synthetic construct
  source 1..35: mol_type = other DNA
tatatttgcaaatgcaattctcaaaaagaggtctt

SEQ ID NO: 37 - DNA, 35 bp, synthetic construct
  source 1..35: mol_type = other DNA
gtaccaacattaacatataatccaacaggcggaat

SEQ ID NO: 38 - DNA, 35 bp, synthetic construct
  source 1..35: mol_type = other DNA
gaaccagattgaatcgtttactcaggacagtgaat

SEQ ID NO: 39 - DNA, 35 bp, synthetic construct
  source 1..35: mol_type = other DNA
cgaaagaaatgaccaggaattaacggaaggatatt

SEQ ID NO: 40 - DNA, 35 bp, synthetic construct
  source 1..35: mol_type = other DNA
gagcaactctacgtacgtaacaatcatacctaaaa

SEQ ID NO: 41 - DNA, 35 bp, synthetic construct
  source 1..35: mol_type = other DNA
atgcagagtagaaaagagtaaatgaacgattaaac

SEQ ID NO: 42 - DNA, 35 bp, synthetic construct
  source 1..35: mol_type = other DNA
aaccaaaagaactgcgagaaaacctgcttttgacc

SEQ ID NO: 43 - DNA, 28 bp, synthetic construct
  source 1..28: mol_type = other DNA
cggaacggaatacaccgctttttatcag

SEQ ID NO: 44 - DNA, 28 bp, synthetic construct
  source 1..28: mol_type = other DNA
accaactatgccactcggaactccaaaa

SEQ ID NO: 45 - DNA, 35 bp, synthetic construct
  source 1..35: mol_type = other DNA
cctcagcggagcctattttctgagccacctcagta

SEQ ID NO: 46 - DNA, 35 bp, synthetic construct
  source 1..35: mol_type = other DNA
ctacagaaatttttagcggagccgccacagtatag

SEQ ID NO: 47 - DNA, 35 bp, synthetic construct
  source 1..35: mol_type = other DNA
aggcttggaatttcacgatctacccatgctgagac

SEQ ID NO: 48 - DNA, 28 bp, synthetic construct
  source 1..28: mol_type = other DNA
tttccaggggatagattaggataagttt

SEQ ID NO: 49 - DNA, 28 bp, synthetic construct
  source 1..28: mol_type = other DNA
taaacaaccctcaggccgtcggcgtcat

SEQ ID NO: 50 - DNA, 35 bp, synthetic construct
  source 1..35: mol_type = other DNA
aaattattcattaaaaccgcctaccgaagaaacaa

SEQ ID NO: 51 - DNA, 28 bp, synthetic construct
  source 1..28: mol_type = other DNA
gtttagtatgagaatagaaagggaattg

SEQ ID NO: 52 - DNA, 48 bp, synthetic construct
  source 1..48: mol_type = other DNA
ctgaattacaaacacctttagaccatcgatagcagcaccgtacagaat

SEQ ID NO: 53 - DNA, 28 bp, synthetic construct
  source 1..28: mol_type = other DNA
aatcctgtgcccttcaccgcctcgctca

SEQ ID NO: 54 - DNA, 20 bp, synthetic construct
  source 1..20: mol_type = other DNA
gaaaatacatacattacgca

SEQ ID NO: 55 - DNA, 20 bp, synthetic construct
  source 1..20: mol_type = other DNA
caatagcaagcaaaatcgta

SEQ ID NO: 56 - DNA, 20 bp, synthetic construct
  source 1..20: mol_type = other DNA
aattacctgagcaacgcaga

SEQ ID NO: 57 - DNA, 20 bp, synthetic construct
  source 1..20: mol_type = other DNA
aatacctacattttgcaaca

SEQ ID NO: 58 - DNA, 28 bp, synthetic construct
  source 1..28: mol_type = other DNA
tgccagctgcattactttccagtcggga

SEQ ID NO: 59 - DNA, 22 bp, synthetic construct
  source 1..22: mol_type = other DNA
agattcaaacgttaatattttg

SEQ ID NO: 60 - DNA, 22 bp, synthetic construct
  source 1..22: mol_type = other DNA
ttcaactataccctgactatta

SEQ ID NO: 61 - DNA, 22 bp, synthetic construct
  source 1..22: mol_type = other DNA
cgaataatggctttgaggacta

SEQ ID NO: 62 - DNA, 22 bp, synthetic construct
  source 1..22: mol_type = other DNA
caagtttgaataaatcctcatt

SEQ ID NO: 63 - DNA, 50 bp, synthetic construct
  source 1..50: mol_type = other DNA
caattccacacaacagctgattttgatggctaaatcagcgaaatgagaag

SEQ ID NO: 64 - DNA, 43 bp, synthetic construct
  source 1..43: mol_type = other DNA
ccggaagcataaagttcaccaggcaaaatgggtcgaggcgctg

SEQ ID NO: 65 - DNA, 50 bp, synthetic construct
  source 1..50: mol_type = other DNA
cctggggtgcctaatgcgccagaagaatatcacccacacgctgccgatta

SEQ ID NO: 66 - DNA, 43 bp, synthetic construct
  source 1..43: mol_type = other DNA
gctaactcacattaagagaggctgagtgtgatggccacccgcc

SEQ ID NO: 67 - DNA, 50 bp, synthetic construct
  source 1..50: mol_type = other DNA
tgcgctcactgcccgatgaatcacaagagcgaaaaagctacaggtgcttt

SEQ ID NO: 68 - DNA, 36 bp, synthetic construct
  source 1..36: mol_type = other DNA
taacaacccacgttggtgtagattaagttgggtaac

SEQ ID NO: 69 - DNA, 43 bp, synthetic construct
  source 1..43: mol_type = other DNA
tgtgctgcaaggcgatgggcgcaaatgtgaaaactatagctat

SEQ ID NO: 70 - DNA, 29 bp, synthetic construct
  source 1..29: mol_type = other DNA
cgccagctggcgaaatctgccatagccag

SEQ ID NO: 71 - DNA, 43 bp, synthetic construct
  source 1..43: mol_type = other DNA
gtgcgggcctcttcgcgacagtaattcgcaaaagccgatattc

SEQ ID NO: 72 - DNA, 29 bp, synthetic construct
  source 1..29: mol_type = other DNA
aactgttgggaaggggatcgcaaatagga

SEQ ID NO: 73 - DNA, 35 bp, synthetic construct
  source 1..35: mol_type = other DNA
ggtgccgaagcgccattcgccattcaggccggcac

SEQ ID NO: 74 - DNA, 17 bp, synthetic construct
  source 1..17: mol_type = other DNA
tcaaccgattgagggag

SEQ ID NO: 75 - DNA, 18 bp, synthetic construct
  source 1..18: mol_type = other DNA
gctatcttccctcagagc

SEQ ID NO: 76 - DNA, 32 bp, synthetic construct
  source 1..32: mol_type = other DNA
aaccttgcttctgtgaaataaagatggtttaa

SEQ ID NO: 77 - DNA, 18 bp, synthetic construct
  source 1..18: mol_type = other DNA
ttcctgtagtcacgacgt

SEQ ID NO: 78 - DNA, 38 bp, synthetic construct
  source 1..38: mol_type = other DNA
aacaagattctccgggtttgcagagagttgcagcaagc

SEQ ID NO: 79 - DNA, 38 bp, synthetic construct
  source 1..38: mol_type = other DNA
cgcctgagggcttgagaaattaattatttgcacgtaaa

SEQ ID NO: 80 - DNA, 42 bp, synthetic construct
  source 1..42: mol_type = other DNA
aattttaatcccatacaaactgtagcattccacaggttgcgc

SEQ ID NO: 81 - DNA, 42 bp, synthetic construct
  source 1..42: mol_type = other DNA
aaacacccgacaatcatcgcccacgcatgaaacaataattaa

SEQ ID NO: 82 - DNA, 42 bp, synthetic construct
  source 1..42: mol_type = other DNA
ggaatcaataaggcgttaaattcgctatagtacaagtatcat

SEQ ID NO: 83 - DNA, 49 bp, synthetic construct
  source 1..49: mol_type = other DNA
taatagattgccagtagtaaaatgtttataattgcttttgaacgtaaga

SEQ ID NO: 84 - DNA, 35 bp, synthetic construct
  source 1..35: mol_type = other DNA
gagcactacaatattcctttttcatttttgcggat

SEQ ID NO: 85 - DNA, 42 bp, synthetic construct
  source 1..42: mol_type = other DNA
atacgtgccatcacgaattaggcaataaagcctcatatcagg

SEQ ID NO: 86 - DNA, 35 bp, synthetic construct
  source 1..35: mol_type = other DNA
cgaacgtgatttagagcttgaccacgcttgggaac

SEQ ID NO: 87 - DNA, 28 bp, synthetic construct
  source 1..28: mol_type = other DNA
attgaccgtaatggttttcccgtgaaat

SEQ ID NO: 88 - DNA, 49 bp, synthetic construct
  source 1..49: mol_type = other DNA
aggcaaagcaaattcaccgagtaaaagaaagccggtcattgctggagca

SEQ ID NO: 89 - DNA, 35 bp, synthetic construct
  source 1..35: mol_type = other DNA
actttaatcattgtagaagttttagagctctttag

SEQ ID NO: 90 - DNA, 35 bp, synthetic construct
  source 1..35: mol_type = other DNA
tgaaataatcttaccccctgcaacctattattctg

SEQ ID NO: 91 - DNA, 28 bp, synthetic construct
  source 1..28: mol_type = other DNA
caccagtcctaatttcctgaacaagaaa

SEQ ID NO: 92 - DNA, 35 bp, synthetic construct
  source 1..35: mol_type = other DNA
ccctcagaaccgccttaatgccaacgctcagctac

SEQ ID NO: 93 - DNA, 28 bp, synthetic construct
  source 1..28: mol_type = other DNA
aacgagcagagcaagccctttgcgacat

SEQ ID NO: 94 - DNA, 35 bp, synthetic construct
  source 1..35: mol_type = other DNA
gtgataataattacagataagtacgagcttgcacc

SEQ ID NO: 95 - DNA, 35 bp, synthetic construct
  source 1..35: mol_type = other DNA
cgtcaatatatcaagcgtagaagtgaatttttccc

SEQ ID NO: 96 - DNA, 35 bp, synthetic construct
  source 1..35: mol_type = other DNA
gtgaggcaaccgttctgaaagtggctattaaaata

SEQ ID NO: 97 - DNA, 35 bp, synthetic construct
  source 1..35: mol_type = other DNA
tgttatcggccctgcccagcaagcccccggcgaga

SEQ ID NO: 98 - DNA, 21 bp, synthetic construct
  source 1..21: mol_type = other DNA
gccaggggataggtcgtcgga

SEQ ID NO: 99 - DNA, 35 bp, synthetic construct
  source 1..35: mol_type = other DNA
gaatcgacaaaggcgagcataacaggcaggcttag

SEQ ID NO: 100 - DNA, 35 bp, synthetic construct
  source 1..35: mol_type = other DNA
gtaccttgactggattgcaaagaattacgtaaatt

SEQ ID NO: 101 - DNA, 28 bp, synthetic construct
  source 1..28: mol_type = other DNA
taaattgcaagcgcaaccgataccgata

SEQ ID NO: 102 - DNA, 28 bp, synthetic construct
  source 1..28: mol_type = other DNA
acagcccgtttcgtaaacatgtaaacag

SEQ ID NO: 103 - DNA, 21 bp, synthetic construct
  source 1..21: mol_type = other DNA
accctcaccaccggaggtgaa

SEQ ID NO: 104 - DNA, 41 bp, synthetic construct
  source 1..41: mol_type = other DNA
gcattagtccttataaaggtggcaacatataaaagggcatg

SEQ ID NO: 105 - DNA, 28 bp, synthetic construct
  source 1..28: mol_type = other DNA
attaagacacgggagaattaacaacgtc

SEQ ID NO: 106 - DNA, 28 bp, synthetic construct
  source 1..28: mol_type = other DNA
aaaaatgatagaaggcttatccaagcaa

SEQ ID NO: 107 - DNA, 28 bp, synthetic construct
  source 1..28: mol_type = other DNA
gccgttttgaatataaagtaccaatcgc

SEQ ID NO: 108 - DNA, 28 bp, synthetic construct
  source 1..28: mol_type = other DNA
catatttatccaatcgcaagacgagact

SEQ ID NO: 109 - DNA, 28 bp, synthetic construct
  source 1..28: mol_type = other DNA
accttttttgatgaaacaaacagaatac

SEQ ID NO: 110 - DNA, 28 bp, synthetic construct
  source 1..28: mol_type = other DNA
caagttactatcatcatattccaatttt

SEQ ID NO: 111 - DNA, 28 bp, synthetic construct
  source 1..28: mol_type = other DNA
aaaagttttctaaagcatcacctgaggc

SEQ ID NO: 112 - DNA, 28 bp, synthetic construct
  source 1..28: mol_type = other DNA
ggtcagtaaatcgtctgaaatgacaata

SEQ ID NO: 113 - DNA, 28 bp, synthetic construct
  source 1..28: mol_type = other DNA
ttaccgcccctcgttagaatcagcgctt

SEQ ID NO: 114 - DNA, 41 bp, synthetic construct; identical to SEQ ID NO: 104
  source 1..41: mol_type = other DNA

SEQ ID NO: 115 - DNA, 28 bp, synthetic construct
  source 1..28: mol_type = other DNA
aatgcgccccgtctatcagggctgttcc

SEQ ID NO: 116 - DNA, 28 bp, synthetic construct
  source 1..28: mol_type = other DNA
agtttggaggccaacgcgcgggttgcgt

SEQ ID NO: 117 - DNA, 28 bp, synthetic construct
  source 1..28: mol_type = other DNA
ttttaaccctccagccagctttctgcgc

SEQ ID NO: 118 - DNA, 28 bp, synthetic construct
  source 1..28: mol_type = other DNA
gccggagaaagcaaatatttaactcatt

SEQ ID NO: 119 - DNA, 28 bp, synthetic construct
  source 1..28: mol_type = other DNA
aacctgttttttagaaccctcaagaaag

SEQ ID NO: 120 - DNA, 28 bp, synthetic construct
  source 1..28: mol_type = other DNA
aggaagcctaacagttgattccgtcaat

SEQ ID NO: 121 - DNA, 28 bp, synthetic construct
  source 1..28: mol_type = other DNA
catagtaagttcagaaaacgagcttcaa

SEQ ID NO: 122 - DNA, 28 bp, synthetic construct
  source 1..28: mol_type = other DNA
caagaacccaacattattacagtacata

SEQ ID NO: 123 - DNA, 28 bp, synthetic construct
  source 1..28: mol_type = other DNA
aatacgtattgaaagaggacagtcttga

SEQ ID NO: 124 - DNA, 28 bp, synthetic construct
  source 1..28: mol_type = other DNA
tgaaaatcgagggtagcaacgggggtaa

SEQ ID NO: 125 - DNA, 28 bp, synthetic construct
  source 1..28: mol_type = other DNA
aaccgccactttcaacagtttctcacgt

SEQ ID NO: 126 - DNA, 28 bp, synthetic construct
  source 1..28: mol_type = other DNA
tccagtaaagagggttgatatacctcag

SEQ ID NO: 127 - DNA, 28 bp, synthetic construct
  source 1..28: mol_type = other DNA
actgtagctggccttgatattctaccgt

SEQ ID NO: 128 - DNA, 41 bp, synthetic construct
  source 1..41: mol_type = other DNA
agacgatgcgttttgccggaaacgtcaccaatgaacgtcag

SEQ ID NO: 129 - DNA, 37 bp, synthetic construct
  source 1..37: mol_type = other DNA
tttttttttttttttccggaagcataaagttcaccag

SEQ ID NO: 130 - DNA, 21 bp, synthetic construct
  source 1..21: mol_type = other DNA
gcaaaatgggtcgaggcgctg

SEQ ID NO: 131 - DNA, 37 bp, synthetic construct
  source 1..37: mol_type = other DNA
cccaaaaaaaaaaaatgtgctgcaaggcgatgggcgc

SEQ ID NO: 132 - DNA, 21 bp, synthetic construct
  source 1..21: mol_type = other DNA
aaatgtgaaaactatagctat

SEQ ID NO: 133 - DNA, 28 bp, synthetic construct
  source 1..28: mol_type = other DNA
ccttgagagagccgaaatcacgacttga

SEQ ID NO: 134 - DNA, 35 bp, synthetic construct
  source 1..35: mol_type = other DNA
cccaaaaaaaaaaaagccatttgggaatttctttt

SEQ ID NO: 135 - DNA, 35 bp, synthetic construct
  source 1..35: mol_type = other DNA
gttcggaaaccatatgaattaaaaaaaaaaaaaaa

SEQ ID NO: 136 - DNA, 37 bp, synthetic construct
  source 1..37: mol_type = other DNA
ctggtgaactttatgcttccggaaaaaaaaaaaaaaa

SEQ ID NO: 137 - DNA, 28 bp, synthetic construct
  source 1..28: mol_type = other DNA
aacagcctcagagacagaaggaatagaa

SEQ ID NO: 138 - DNA, 35 bp, synthetic construct
  source 1..35: mol_type = other DNA
tttttttttttttttaattcatatggtttccgaac